(19) United States
(12) Patent Application Publication
(10) Pub. No.: US 2026/0083581 A1
(43) Pub. Date: Mar. 26, 2026

(54) MEDICAL BRACE AND METHODS OF PREPARATION AND USE THEREOF

(71) Applicant: John Noel, Las Cruces, NM (US)

(72) Inventor: John Noel, Las Cruces, NM (US)

(21) Appl. No.: 19/339,908

(22) Filed: Sep. 25, 2025

Related U.S. Application Data
(60) Provisional application No. 63/699,431, filed on Sep. 26, 2024.

(51) Int. Cl. A61F 5/01

(52) U.S. Cl. CPC A61F 5/0104

(57) ABSTRACT

The disclosure relates to a brace, methods of preparation, and methods of using the brace. The brace comprises a plurality of first helix structures and a plurality of second helix structures. Each first helix winds in a first direction from a proximal end to a distal end, conforming to a contour of an object. Each second helix winds in a second direction from the proximal end to the distal end, also conforming to the contour of the object. Each first helix of the plurality of first helix structures is rotatably coupled to a corresponding second helix of the plurality of second helix structures, forming a helix pair. The first direction of the plurality of first helix structures opposes the second direction of the plurality of second helix structures.

### RELATED APPLICATIONS

This application claims the benefit of U.S. Provisional Application No. 63/699,431, filed on Sep. 26, 2024, the entire contents of which are incorporated by reference herein in its entirety.

### TECHNICAL FIELD

The disclosure relates to medical devices (e.g., braces) and methods of preparation (e.g., three-dimensional printing) and use thereof.

### BACKGROUND

Medical bracing solutions represent a significant segment of the general orthopedic product market, currently an $11.8 billion industry in the United States alone. Current medical bracing relies on standardized, mass-produced devices that are manufactured using traditional molding and fabrication techniques. These conventional braces are constructed from rigid materials and employ a one-size-fits-all approach, with standard sizes produced in bulk quantities. The manufacturing process involves extensive material usage, often resulting in significant waste, and relies on molds and templates that cannot be customized.

This standardized approach creates interconnected problems that compromise patient outcomes and system efficiency. The one-size-fits-all methodology results in improper fitting, as individual anatomical variations cannot be accommodated within predetermined size categories. These ill-fitting devices provide inadequate support and cause discomfort through pressure points, chafing, and restricted movement. The rigid construction materials result in heavy, inflexible devices that patients find cumbersome in daily activities. Consequently, patient compliance suffers significantly, with many individuals—including injured athletes and elderly patients—abandoning their prescribed braces or seeking non-medical alternatives. This can potentially lead to further injury or delayed healing.

Current manufacturing processes further compound these issues through economic and environmental inefficiencies. Traditional production methods involve substantial material waste, costly tooling, and environmentally unsustainable practices that increase healthcare system costs. The combination of poor patient compliance, inadequate therapeutic outcomes, and inefficient production highlights the need for improved medical devices tailored to individual anatomical requirements, along with more sustainable manufacturing processes that enhance patient recovery while reducing healthcare costs and environmental impact.

### BRIEF DESCRIPTION OF THE DRAWINGS

Aspects and implementations of the present disclosure will be understood more fully from the detailed description below and the accompanying drawings of various aspects and implementations of the disclosure, which, however, should not be taken to limit the disclosure to the specific aspects or implementations, but are for explanation and understanding only.

FIG. 1 illustrates a back perspective view of a brace, according to at least one embodiment.

FIG. 2 illustrates a top perspective view of the brace of FIG. 1 shown in a vertical orientation, according to at least one embodiment.

FIG. 3 illustrates a bottom perspective view of the brace of FIG. 1, according to at least one embodiment.

FIG. 4 illustrates a left perspective view of the brace of FIG. 1 shown in a horizontal orientation, according to at least one embodiment.

FIG. 5 illustrates a right perspective view of the brace of FIG. 1 shown in a horizontal orientation, according to at least one embodiment.

FIG. 6 illustrates a right perspective view of the brace of FIG. 1 shown in an inverted orientation from FIG. 1, according to at least one embodiment.

FIG. 7 illustrates a front perspective view of the brace of FIG. 1 shown in a vertical orientation, according to at least one embodiment.

FIG. 8 is an exemplary computer system used to generate the brace of FIG. 1, according to at least one embodiment.

FIG. 9 is a graphical representation of a movement of an object along its medial axis, according to at least one embodiment.

FIG. 10 is a graphical representation of a sigmoid curve, according to at least one embodiment.

FIG. 11 is a graphical representation of a tilt of a helix with respect to a reference axis, according to at least one embodiment.

FIG. 12 is a block diagram of an example computer system, according to at least one embodiment.

### DETAILED DESCRIPTION

The present disclosure introduces a brace that provides significant advantages over traditional orthopedic supports and is adaptable for both medical and non-medical applications. Featuring a helically wound design, the brace offers a custom, form-fitting structure that improves comfort, mobility, and user independence. Its lightweight, breathable, and flexible construction promotes long-term wear, while advanced fabrication methods enable rapid and precise customization for each user or object, thereby reducing costs and supporting sustainability.

The brace is designed to conform to the contours of various objects, including body parts, prosthetic limbs, medical devices, sports equipment, tools, packages, and electronic devices. It consists of multiple first (e.g., dextrorotatory) and second (e.g., levorotatory) helices, each winding in opposite directions from a proximal end to a distal end, which are rotatably coupled to form a pair of helix structures (e.g., helix pairs). These helices overlap in a crosshatched configuration, creating a network of openings that dynamically respond to axial compression or extension by adjusting the distance between heir proximal and distal ends.

The formed helix pairs are joined using a pin-and-hole connection, which allows for relative rotation within each helix pair. Customization is achieved by determining the shape of the object, selecting appropriate helix dimensions, and configuring the helices to follow and maintain the object's contours. The brace can be constructed from a wide range of materials, including polymers, metals, ceramics, fabrics, natural fibers, synthetic fibers, and elastomers. Optional additives may be incorporated to enhance properties such as waterproofing, temperature resistance, antimicrobial effects, UV protection, and coloration. The helices themselves may be formed from units such as particles, granules, extrudates, fibers, or powders.

The present disclosure also encompasses a method for designing and manufacturing the brace, which involves determining the object's contours, selecting appropriate helix dimensions, configuring and orienting the helices, and operatively coupling them to ensure the brace conforms to and adapts with the object's shape. This structure offers a versatile, adaptable, and resilient bracing solution suitable for a variety of applications, such as reinforcing delicate equipment, securing valuables during transport, or stabilizing components across different industries. The combination of a custom, dynamically supportive structure and advanced fabrication methods highlights the broad potential of this technology to provide protective and supportive solutions for a wide range of objects and environments.

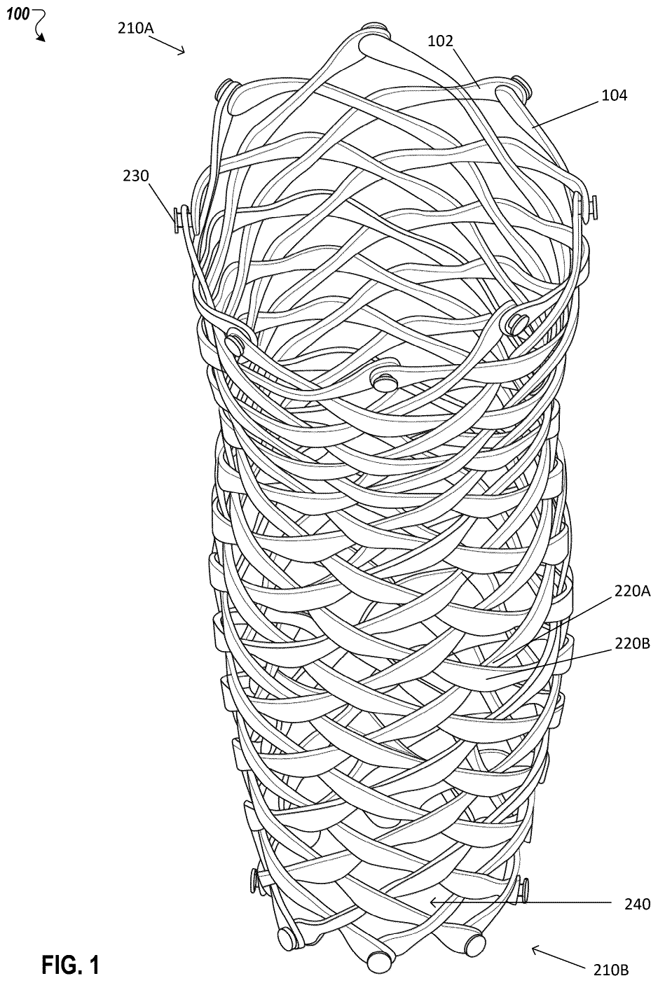

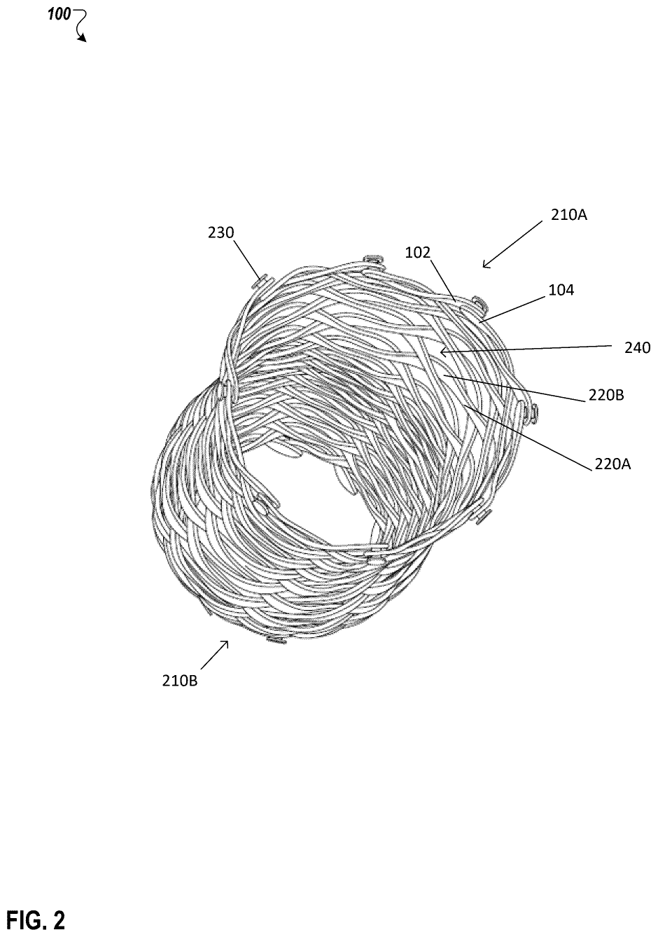

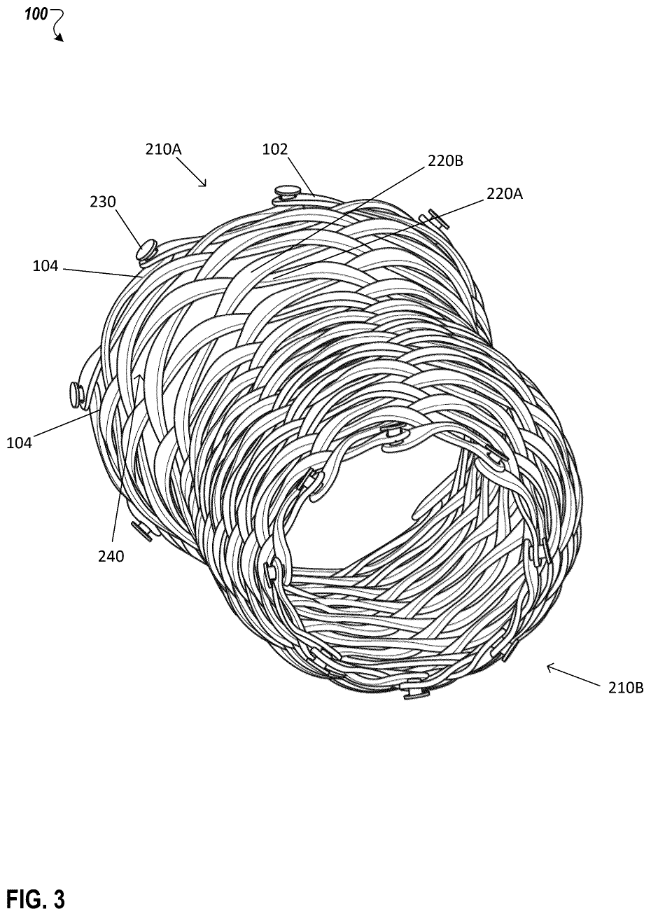

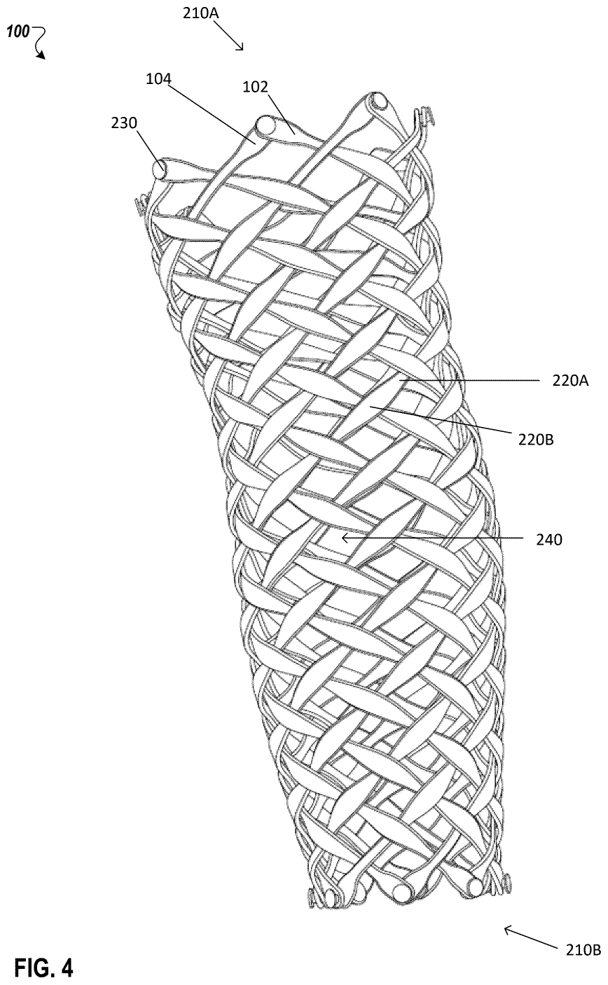

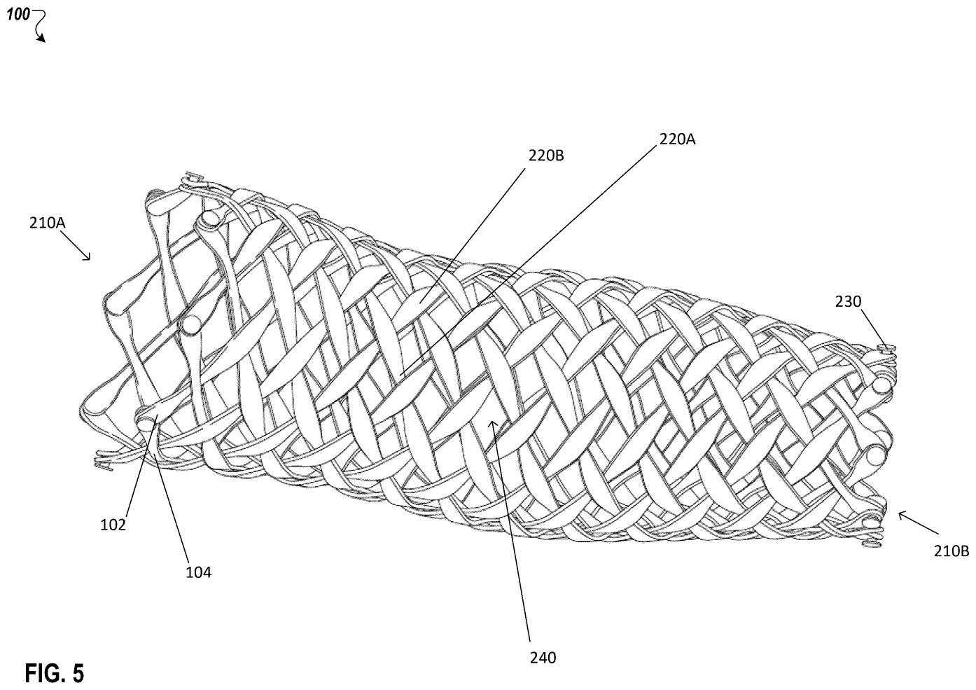

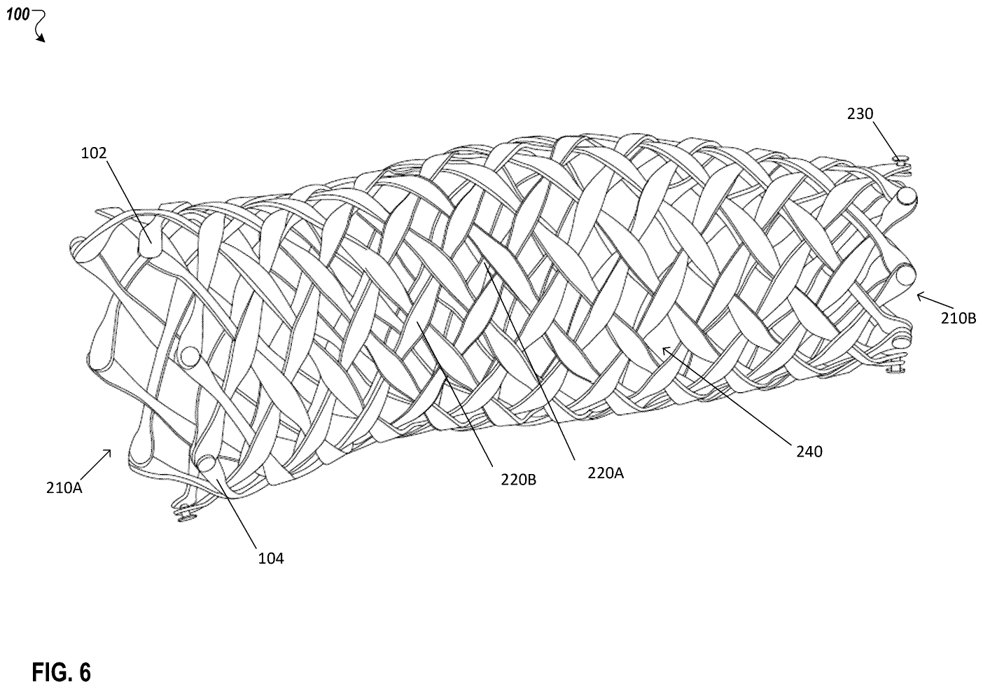

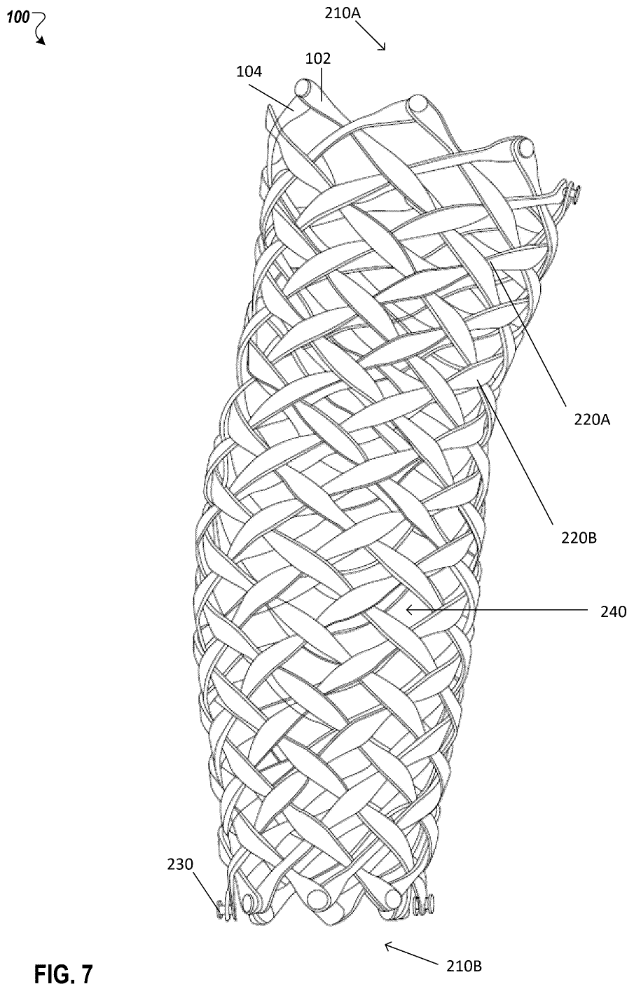

FIGS. 1-7 illustrate various perspectives of a brace 100. Brace 100 includes a plurality of helix structure (e.g., plurality of helices), each of which is either a dextrorotatory helix structure (e.g., dextrorotatory helix 102) or a levorotatory helix structure (e.g., levorotatory helix 104). The dextrorotatory helix (e.g., dextrorotatory helix 102) winds in a clockwise direction from a proximal end 210A to a distal end 210B, while the levorotatory helix (e.g., levorotatory helix 104) winds in a counterclockwise direction from the proximal end 210A to the distal end 210B. In other words, the dextrorotatory and levorotatory helices wind in opposite directions, both starting from the proximal end 210A of the brace 100 and extending to the distal end 210B. Each helix is constructed so that a predefined profile is consistently maintained along its entire length. This means the profile at every point remains unchanged, ensuring that each helix retains uniformity in shape throughout its entire configuration. The predefined profile may include, but is not limited to, a rectangular, circular, elliptical, or any other suitable geometric shape.

Each helix of the brace 100 may include one or more regions of varying width. For example, in some regions, the width of the helix is tapered (e.g., tapered region 220A), while in other regions, the width is flared (e.g., flared region 220B). Tapered regions (e.g., tapered region 220A) and flared regions (e.g., flared region 220B) of each helix alternate to permit overlapping. For instance, the tapered regions of the dextrorotatory helix allow overlapping with the flared regions (e.g., flared region 220B) of the levorotatory helix, and vice versa. Additionally, in some embodiments, the flared regions (e.g., flared region 220B) of each helix are elevated above the predefined profile, while the tapered regions (e.g., tapered region 220A) are recessed below the predefined profile. As a result, the dextrorotatory helix (e.g., dextrorotatory helix 102) and the levorotatory helix (e.g., levorotatory helix 104) do not come into contact with one another along their length, allowing for movement.

In some embodiments, the dextrorotatory helix (e.g., dextrorotatory helix 102) and the levorotatory helix (e.g., levorotatory helix 104) can be rotatably coupled to form a helix pair. Each dextrorotatory helix and levorotatory helix may be joined using a pin-and-hole connection, which allows for rotational movement. For example, the dextrorotatory helix may include a hole (not shown) designed to receive a pin (e.g., pin 230) that is fixed to the levorotatory helix. The pin may feature an enlarged head to prevent accidental disengagement, ensuring that the dextrorotatory and levorotatory helices of each helix pair remain securely connected and properly aligned. Additionally, the pin-and-hole connection can be configured to permit rotation of the dextrorotatory helix (or first helix) and the levorotatory helix (or second helix) of a respective helix pair about the pin. This allows the helices to pivot relative to one another while remaining reliably engaged during use. In some embodiments, the proximal end 210A and the distal end 210B of each helix may be rounded to further facilitate rotation, thereby reducing friction and enabling smoother relative movement.

Each formed helix pair interlaces in a helical or crosshatched configuration between the proximal end 210A and the distal end 210B, creating a network of openings (e.g., opening 240). Each opening in this network allows for breathability, as well as expansion and contraction, due to the operative coupling of the dextrorotatory helix (e.g., dextrorotatory helix 102) and the levorotatory helix (e.g., levorotatory helix 104) that form each helix pair.

The shape and size of each opening in the network can be adjusted in response to forces applied to the proximal end 210A and the distal end 210B. When a force is applied to the proximal end 210A toward the distal end 210B, and a simultaneous force is applied to the distal end 210B toward the proximal end 210A (e.g., axial compression on the brace 100), resulting in a decrease in the distance between the proximal end 210A and distal end 210B of each opening (e.g., opening 240). Conversely, when a force is applied to the proximal end 210A away from the distal end 210B, and a simultaneous force is applied to the distal end 210B away from the proximal end 210A (e.g., axial extension on the brace 100), increasing the distance between the proximal end 210A and distal end 210B of each opening. This axial movement of the brace 100 converts externally applied tension forces into internal compression forces within the brace.

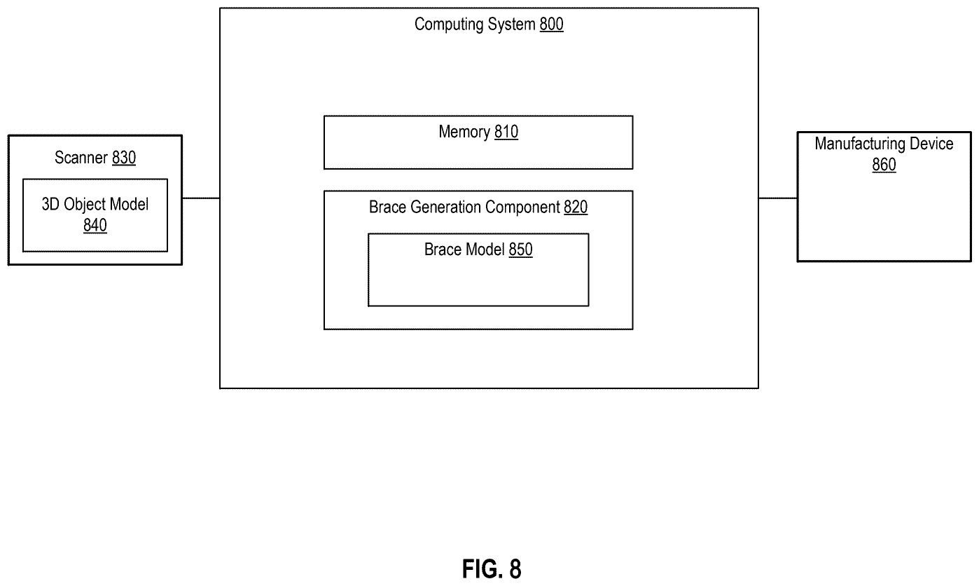

FIG. 8 illustrates an example computing system 800 that includes a brace generation component used to generate a customized brace (similar to brace 100 of FIGS. 1-7) in accordance with one or more aspects of the present disclosure.

In some embodiments, various parameters of the brace 100 of FIG. 1 may be dynamically generated and customized to conform to and support an object, including accommodating the anatomical or structural contours of that object. For example, the brace 100 may be computationally synthesized based on a scan of the object to ensure a secure and tailored fit. The brace 100 may be designed for a wide variety of objects, including but not limited to body parts, prosthetic limbs, medical devices (e.g., catheters, tubes), sports equipment (e.g., paddles, bats), tools or instruments, packages or fragile items, furniture components, and electronic devices. Body parts may include, but are not limited to, knees, ankles, wrists, elbows, shoulders, backs (or spines), necks, hands, fingers, feet, toes, hips, chests, arms, legs, ribs, joints, shins, calves, thighs, biceps, triceps, forearms, lower back, or head. In other words, the brace 100 may be synthesized and adapted for a wide range of medical and non-medical applications where structural support, stabilization, or protection is required.

The computing system 800 may be any processing environment configured to execute instructions, manage system operations, or interface with other components of the system described herein. In one or more embodiments, the computing system 800 may include one or more processors, such as central processing units (CPUs), graphics processing units (GPUs), digital signal processors (DSPs), field-programmable gate arrays (FPGAs), or application-specific integrated circuits (ASICs), configured to perform parallel or sequential computations. The computing system 800 may also include communication interfaces for wired or wireless connectivity, input/output (I/O) interfaces for peripheral communication, and software or firmware modules for executing control logic, signal processing, data analytics, or machine learning tasks. In some embodiments, the computing system 800 may be implemented as a local workstation, a cloud-based platform, an embedded processor in a peripheral device, or a distributed system composed of multiple networked computing nodes.

The computing system 800 may include a memory 810. The memory 810 may be any non-transitory computer-readable medium configured to store data and executable instructions for use by the computing system 800. In various embodiments, the memory 810 may include one or more types of storage, such as dynamic or static random-access memory (DRAM, SRAM), read-only memory (ROM), flash memory, magnetic disks, solid-state drives (SSDs), or optical storage media. The memory 810 may store software applications, operating system components, control algorithms, configuration data, 3D models, intermediate processing results, or machine learning parameters, depending on the operational requirements of the system. In some embodiments, the memory 810 may be partitioned into volatile and non-volatile regions to support both runtime execution and persistent storage. Additionally, the memory 810 may facilitate communication between system components by buffering data exchanged between scanning devices, additive manufacturing devices, and user interfaces.

The brace generation component 820 may receive a request to generate a brace (e.g., brace 100 of FIGS. 1-7) for an object. The brace generation component 820 scans the object using a scanner 830. The scanner 830 generates a 3D image of the object (not shown). Specifically, the scanner 830 establishes a coordinate reference system for the scanning environment and systematically captures spatial data points across the surface of the object from multiple angles or positions. The scanner 830 then registers and aligns the captured data points to create a unified point cloud, processes the point cloud data to remove noise and fill gaps, and generates a 3D model representing the geometric properties and surface characteristics of the object (e.g., 3D object model 840). The scanner 830 provides the 3D object model 840 to the brace generation component 820, which stores it in the memory 810.

The scanner 830 may be any device capable of capturing three-dimensional (3D) spatial data of an object, including but not limited to optical scanning devices such as structured light scanners, laser triangulation scanners, and photogrammetry systems; time-of-flight devices such as light detection and ranging (LIDAR) systems and time-of-flight cameras; stereo vision systems employing multiple cameras for depth perception; contact-based coordinate measuring machines; or combinations thereof. In some embodiments, photogrammetry techniques may be implemented using camera systems operated by application software on computing devices (e.g., mobile phones) to reconstruct 3D models from multiple two-dimensional images. In other embodiments, LIDAR systems utilize laser pulses to measure distances and generate point cloud data for 3D reconstruction of the model. Structured light scanners project known light patterns onto the surface of the object and analyze the deformation of these patterns through camera systems to calculate the surface geometry for the 3D model. In various embodiments, the scanning device may incorporate machine learning algorithms for enhanced data processing, noise reduction, and automated feature recognition during the 3D reconstruction process.

The brace generation component 820 uses the 3D object model 840 to generate a brace model 850. Specifically, the brace generation component 820 computes a medial axis from the 3D object model 840. The medial axis is defined as the set of points equidistant from the boundaries of the 3D object model 840 and serves as a central path for brace construction. To achieve this, the brace generation component 820 employs techniques such as piecewise linear interpolation across segmented cross-sections, 3D feature recognition of principal axes, and the medial axis transform (MAT), which produces a centerline for arbitrary geometries.

The brace generation component 820 also determines the kinematics of the medial axis derived from the 3D object model 840. In some embodiments, the brace generation component 820 may implement the medial axis transform (MAT) using a C++ executable built with the CGAL library, enabling the computation of central axes from arbitrary 3D object geometries. The brace generation component 820 calculates the vector position p(t) along the medial axis as a function of a scalar path variable t and evaluates the corresponding velocity and acceleration. These kinematic quantities allow the component to characterize how the geometry evolves along the axis and to guide conformal helix construction in response to local curvature and shape variation. In particular, motion along the medial axis is described using equation (1):

$$p ( t ) = [ {p}_{i} , … , {p}_{a} ] , v ( t ) = dp / dt , a ( t ) = {d}^{2} p / {dt}^{2} , \qquad ( 1 )$$

where p(t) defines the position vector along the medial axis as a function of path parameter t, v^{→}(t) is the velocity vector (i.e., the rate of change of position), and a^{→}(t) is the acceleration vector (i.e., the rate of change of velocity). These vectors enable the brace generation component 820 to simulate how the geometry of the 3D object model 840 changes along its form, which is essential for adapting the helix to regions with varying curvature or anatomical complexity. As a result, the brace generation component 820 can modify the standard helical vector formula to follow the computed medial axis, producing a conformal brace structure that is precisely tailored to the anatomy of the 3D object model 840.

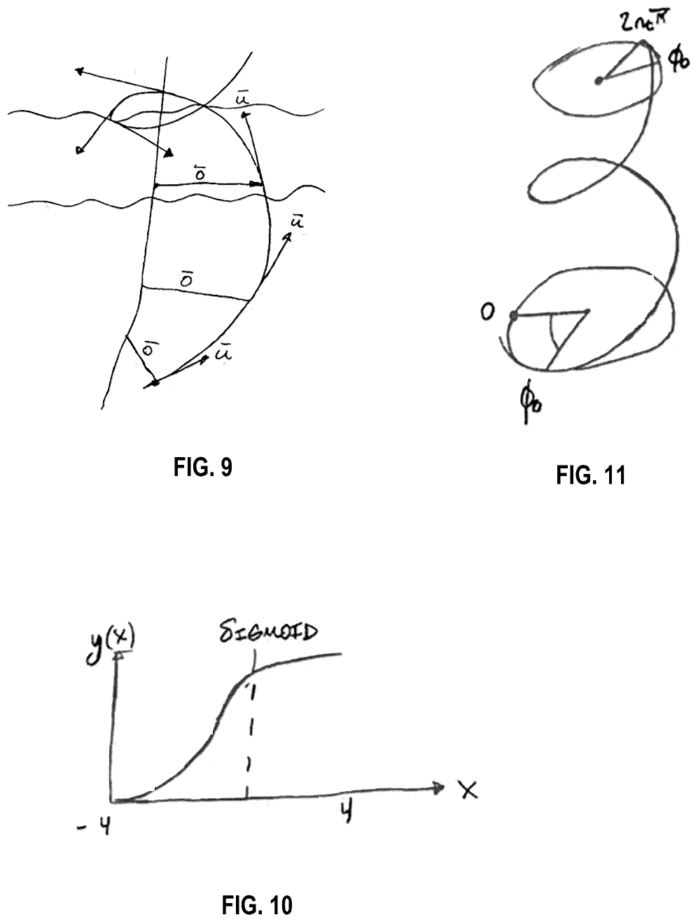

To construct a helix that accurately wraps around the curved medial axis, the brace generation component 820 calculates a direction vector that is orthogonal to the tangent of the medial path. This approach ensures that the helix consistently coils in a perpendicular orientation, even as the path bends or twists. As illustrated in FIG. 9, the tangent-adjusted direction vector u′ is computed using equation (2):

$${u}^{\to } = \hat{a} - {v}^{\to } ( \hat{a} \cdot {v}^{\to } ) , \qquad ( 2 )$$

where â is a reference axis (e.g., the vertical direction), v^{→} is the unit tangent vector along the medial axis, and the dot product (â·v^{→}) projects â onto v^{→}, subtracting the projection yields a vector perpendicular to v^{→}. The component uses u^{→} to initialize the starting orientation of the radius vector around which the helix is wrapped.

Before computing tangent adjustments or determining directional orientation, the brace generation component 820 normalizes all involved vectors to ensure mathematical stability and consistent behavior along the medial axis of the 3D object model 840, as shown in equation (3):

$$\hat{x} = {x}^{\to } / ‖ {x}^{\to } ‖ , \qquad ( 3 )$$

where x^{→} is any vector (e.g., v^{→} or a^{→}), \hat{x} is the corresponding unit vector (direction with magnitude 1), and ∥x^{→}∥ is the Euclidean norm (vector length). Equation (3) ensures that directional computations, dot products, and vector rotations behave correctly during helix construction. Without normalization, variations in vector magnitudes could distort the orientation or radius of the helix.

The brace generation component 820 uses a scalar path parameter/to represent position along the medial axis, enabling dynamic modeling of brace geometry. The corresponding velocity and acceleration vectors v^{→}(t) and a^{→}(t) describe how the medial path changes, allowing adaptation of orientation and radius without requiring knowledge of a global pose of the 3D object model 840.

The brace generation component 820 constructs a conformal helix (e.g., a helix of brace 100 in FIGS. 1-7) around the medial axis by modifying the standard helical vector formula to account for curvature. The angular orientation of the helix is governed by a constant helix angle do, as shown in FIG. 11 and represented by equation (4):

$$ϕ ( t ) = ( 2 π \cdot ‖ {v}^{\to } ( t ) ‖ ) / pitch , \qquad ( 4 )$$

where pitch refers to the vertical distance between turns of the helix. The circumference corresponds to the radial path length of a full revolution around the medial axis.

The brace generation component 820 computes the radius vector orientation using equation (5):

$${r}^{⋀} = \hat{a} cos ϕ + ( \hat{a} \times {v}^{⋀} ) sin ϕ , \qquad ( 5 )$$

where directional vector \hat{r} defines the local basis for positioning points around the medial axis and modulating the helix geometry.

These calculations-including the tangent-adjusted direction vector u^{→} in Equation (2) to align the helix orthogonally to the medial axis, the normalization of vectors in Equation (3) to ensure consistent directional behavior across the 3D object model 840, and the angular orientation φ(t) in Equation (4) that governs helical rotation based on local path velocity and pitch-allow the conformal helix to wrap smoothly around the medial axis of the 3D object model 840 while maintaining a consistent angular orientation, even as the curvature changes.

The brace generation component 820 performs raycasting operations outward from the medial axis at discrete intervals to determine the local radius profile of the object. By casting rays in the direction of the radius vector r\hat{ }, the brace generation component 820 identifies intersections with the surface of the 3D object model 840 and computes the radial distance from the medial axis to the boundary using Equation (6):

$$R ( p , r \hat{} ) = Raycast {r}^{⋀} from p to the 3 D object model 840 \qquad ( 6 )$$

As a result, the brace generation component 820 constructs, using Equation (6), a medial axis-aligned radius function that maps each position along the medial axis to a corresponding boundary distance. This function captures spatial variations in the thickness of the 3D object model 840.

The brace generation component 820 then uses the resulting radius function to define a conformal brace geometry that varies in radius along the medial path. This enables anatomically accurate fitting or structural conformity tailored to the geometry of the scanned object.

The brace generation component 820 applies a weave function to modulate the helix with oscillatory patterns using equation (7). The weave function enables the formation of intricate, oscillatory patterns along the helical path, inspired by natural braid or textile geometries, thereby adjusting shape complexity using parameters like displacement amplitude and frequency.

$$W = wd cos ( f ϕ ) , \qquad ( 7 )$$

where wd is weave displacement and f is weave frequency. Accordingly, the brace generation component 820 adjusts wd and f to vary the shape, density, and complexity of the woven helix structure for structural or aesthetic outcomes.

The brace generation component 820 evaluates how closely the helix conforms to an ideal spiral using equation (8):

$${R}^{\to } = {p}^{\to } + ( W + O + R ) {r}^{⋀} , \qquad ( 8 )$$

where p^{→} is medial axis position, W is weave component, O is offset distance, R is base radius, and \hat{r} is unit radius vector. Thus, the brace generation component 820 constructs a helix that conforms precisely to the geometry of the 3D object model 840.

The brace generation component 820 modulates structural parameters, such as helix pitch, radius, and thickness, along the medial axis of the 3D object model 840 to enable smooth, spatially adaptive transitions in the brace geometry. This modulation allows the brace to conform to varying anatomical or structural complexity.

In some embodiments, the brace generation component 820 further includes a software-based optimization module that employs machine learning to select, modulate, or predict geometric and process parameters for the brace model 850. Training data may include scans, usage telemetry, and/or outcome labels across multiple object categories (e.g., distinct body parts, prosthetics, tools), enabling the brace generation component 820 to generalize to new objects while preserving object-specific fit. The brace generation component 820 can optimize, singly or jointly, helix pitch, radius, thickness, weave displacement W, weave frequency f, and material/process selections under one or more objective functions, such as fit fidelity, pressure or strain uniformity, predicted comfort and compliance, ventilation area, mass, manufacturability (e.g., print time, support usage), impact energy absorption, or durability. Inference may execute locally on the computing system 800 or remotely via a networked service, and the resulting parameter fields are applied along the medial axis to produce a manufacturing-ready brace model 850 without manual tuning. In some embodiments, the brace generation component 820 uses supervised, unsupervised, or reinforcement learning, optionally with uncertainty estimation, to recommend parameter bounds, detect out-of-distribution geometries, and suggest alternative brace layouts, thereby expanding applicability across multiple objects and use cases.

As shown in FIG. 10, the brace generation component 820 uses a standard sigmoid function and its inverted variant, defined by equations (9) and (10), respectively:

$$y ( x ) = 1 / ( 1 + e \hat{} ( - x ) ) // standard sigmoid \qquad ( 9 )$$

$$y ( x ) = - {( 1 + e \hat{} ( - x ) )}^{- 1 *} // inverted sigmoid variant \qquad ( 10 )$$

The brace generation component 820 maps angular positions along the helix into a normalized sigmoid input domain, enabling smooth interpolation between geometric states. This allows spatially localized transitions in helix pitch, radius, and thickness.

Collectively, the computed medial axis, tangent-adjusted direction vectors, helical orientation and radius vectors, medial axis-aligned radius function, weave-modulated displacement values, and sigmoid-based transition parameters are utilized by the brace generation component 820 to generate the brace model 850 that conforms to the anatomical contours of the scanned object (e.g., the 3D object model 840).

The brace generation component 820 may store the brace model for future use in memory 810 with a unique identifier associated with the scanned object. The brace generation component 820 provides the brace model 850 to a manufacturing device 860 (or in some instances a manufacturer) for manufacturing. As a result, the manufacturing device 860 produces the brace 100 that conforms to the scanned object.

The manufacturing device 860 may be any device capable of producing physical objects from digital or physical design specifications using one or more manufacturing processes. These processes may include, but are not limited to, additive manufacturing, subtractive manufacturing, forming, casting, injection molding, and joining techniques. In one or more embodiments, manufacturing device 860 may implement additive manufacturing (commonly referred to as 3D printing), in which objects are constructed layer by layer from materials such as thermoplastics, resins, or metals. Examples of additive manufacturing systems include fused deposition modeling (FDM) printers that extrude thermoplastic filament through a heated nozzle; stereolithography (SLA) systems that cure photopolymer resin using ultraviolet light; selective laser sintering (SLS) and selective laser melting (SLM) systems that fuse powdered material with lasers; and digital light processing (DLP) devices that use projected light to cure resin layers. In other embodiments, manufacturing device 860 may employ subtractive manufacturing methods, such as CNC machines, mills, lathes, or drills, which remove material from a solid block to form a desired shape. Forming-based manufacturing devices may deform materials through forging, stamping, bending, or rolling. Casting systems may include equipment for pouring molten material into molds for solidification into specific geometries. Injection molding machines may inject molten plastic or other materials into a mold cavity for rapid replication of parts. Joining-based manufacturing devices may include welders, soldering stations, adhesive dispensers, or mechanical assembly tools.

In some embodiments, the manufactured brace incorporates monitoring electronics and/or smart materials to enable real-time sensing, connectivity, and adaptive response. Flexible circuitry, conductive traces, or e-textiles may be integrated into or onto one or more helix structures to host sensors (e.g., strain, pressure, load, temperature, humidity, inertial motion) and communication interfaces (e.g., Near Field Communication (NFC), Bluetooth® Low Energy (BLE), Wi-Fi® (IEEE 802.11), or cellular Internet of Things (IoT)). Power may be supplied by thin-film batteries, inductive charging, or energy harvesting (e.g., piezoelectric or thermoelectric). Sensor data can be encrypted and transmitted to a local device or cloud service for visualization, alerts, or remote adjustment, and can be fed back into the brace generation component 820 to recalibrate model parameters over time (e.g., closed-loop updates to pitch, thickness, or weave density). Smart materials may include piezoresistive or capacitive fabrics for distributed pressure mapping, electroactive polymers for on-command tightening or loosening, and shape-memory alloys or polymers for thermally or electrically actuated fit changes; these materials can be positioned within flared or tapered regions to preserve rotation at pin-and-hole connections. Electronics may be modular or removable to facilitate cleaning or sterilization, and the brace remains fully functional in passive mode when electronics are absent or powered down.

Depending on the type of manufacturing device 860 used and the associated fabrication process, the brace may be constructed using various types of materials that impart different characteristics, including feel, appearance, comfort, flexibility, support, and stability. Suitable materials for constructing the manufactured brace (e.g., brace 100 of FIG. 1) include, but are not limited to, polymers, metals, ceramics, fabrics, natural fibers, synthetic fibers, elastomers, and nanomaterials. These materials may further incorporate one or more additives to enhance the performance or functionality of the manufactured brace, such as thermopolymer additives, waterproof additives, temperature-resistant additives, skin-safe additives, antibacterial additives, antimicrobial additives, ultraviolet (UV) protection additives, colorants, or combinations thereof.

Suitable polymers include, but are not limited to, thermoplastics, polypropylene (PP), acrylonitrile butadiene styrene (ABS), polylactic acid (PLA), acrylic styrene acrylonitrile (ASA), polyamides, nylon, polyethylene terephthalate (PET), polybutylene terephthalate (PBT), polycarbonate (PC), polyether ether ketone (PEEK), polyetherketoneketone (PEKK), polyaryletherketone (PAEK), polyetherimide (PEI), high-impact polystyrene (HIPS), polystyrene (PS), polyvinyl alcohol plastic (PVA), thermoplastic polyurethane (TPU), polyurethane (PU), silicone elastomers, polyethylene (PE), ultra-high molecular weight polyethylene (UHMWPE), thermoplastic elastomers (TPE), polyoxymethylene (POM), polytetrafluoroethylene (PTFE), polyvinylidene fluoride (PVDF), polyphenylsulfone (PPSU), polyphenylene oxide (PPO), liquid crystal polymers (LCP), or combinations of any two or more thereof.

Suitable metals include, but are not limited to, titanium, titanium alloys, aluminum, aluminum alloys, copper, copper alloys, magnesium, magnesium alloys, tungsten, rhenium, niobium, molybdenum, tantalum, gold, platinum, stainless steel, cobalt chrome, cobalt-chromium alloys, steel, carbon steel, nickel, tool steel, nickel-based superalloys (e.g., Inconel 625®), maraging steel, shape memory alloys (e.g., nitinol), zinc, zinc alloys, brass, bronze, silver, palladium, rhodium, iridium, osmium, ruthenium, vanadium, chromium, iron, or combinations or superalloys thereof.

Suitable ceramics include, but are not limited to, alumina (aluminum oxide), silica (silicon dioxide), silicon nitride, silicon carbide, silicon boride, aluminum nitride, zirconia (zirconium dioxide), zirconium silicate, yttria-stabilized zirconia (YSZ), titanium dioxide, titanium carbide, titanium nitride, boron carbide, boron nitride, tungsten carbide, hydroxyapatite, tricalcium phosphate, bioactive glass, mullite, cordierite, spinel, magnesia (magnesium oxide), calcia (calcium oxide), beryllia (beryllium oxide), or combinations thereof.

Suitable fabric materials include, but are not limited to, composite carbon fiber, fiberglass, Kevlar (aramid fiber), carbon-reinforced polyamide (carbon PA), carbon-reinforced polyether ether ketone (carbon PEEK), basalt fiber, ultra-high molecular weight polyethylene (UHMWPE) fabric, polytetrafluoroethylene (PTFE) fabric, polyester fabric, nylon fabric, polypropylene fabric, aramid fabric, woven fabrics, non-woven fabrics, knitted fabrics, braided fabrics, or any combination of two or more thereof.

Suitable natural fibers include, but are not limited to, wood, bamboo, crop straws, flax, jute, hemp, cotton, cellulose derivatives, and combinations of plant- or animal-based fibers such as wool, silk, mohair, or alpaca. In some embodiments, nanocellulose or bacterial cellulose may be included to enhance mechanical strength or bio-integration.

Suitable synthetic fibers include, but are not limited to, aramid, ultra-high molecular weight polyethylene (UHMWPE), nylon, polyester, polypropylene, polyacrylonitrile (PAN), and carbon fibers, or combinations thereof.

Suitable elastomer materials include, but are not limited to, silicone, thermoplastic polyurethane (TPU), natural rubber, styrene-butadiene rubber (SBR), ethylene propylene diene monomer (EPDM), nitrile rubber (NBR), and other synthetic rubbers, or combinations thereof.

Suitable nanomaterials include, but are not limited to, carbon nanotubes, graphene, nanofibers, quantum dots, and nanoparticles of various oxides and metals, as well as polymer-based nanocomposites designed to improve strength, antimicrobial resistance, or biocompatibility.

Suitable additives may be selected based on the intended performance characteristics of the manufactured brace. These may include thermopolymer additives for printability and thermal resistance, waterproof additives to prevent moisture ingress, temperature resistance additives for thermal stability, skin-safe additives for wearer comfort, and antibacterial or antimicrobial additives to prevent infection. Additional additives may include UV-protection additives to reduce degradation due to sunlight exposure and colorants for aesthetic customization.

Additional suitable additive and molding materials include, but are not limited to, 3D printed metals such as AlSi10Mg, bronze-infiltrated stainless alloys (e.g., X1 Metal 420i and 316i), and tungsten-bronze composites, as well as polymers and resins specifically fabricated by additive manufacturing processes, including resins formed via digital light synthesis (DLS), stereolithography (SLA), or PolyJet processes. Further suitable materials include composite-filled variations such as Nylon 12 reinforced with glass beads, Nylon 12 filled with aluminum, or other fiber- or particulate-reinforced polymer formulations that enhance stiffness, dimensional stability, or thermal performance. Additional materials may include cast urethanes, such as rigid urethanes, elastomeric urethanes, and flame-retardant urethanes, as well as silicone elastomers available in a range of durometers, for example TC-5005 Shore A 10 through Smooth-Sil™ 960 Shore A 50-60, or combinations thereof.

In some embodiments, the manufactured brace may include a support structure material comprising a plurality of layers, each bonded, fused, or adhered to at least one adjacent layer to form a cohesive framework. In certain implementations, the manufactured brace may be formed from discrete units such as granules, fibers, filaments, microparticles, or nanoparticles, depending on the chosen fabrication method. The particle or fiber dimensions may be selected to optimize the mechanical, structural, or comfort characteristics of the manufactured brace.

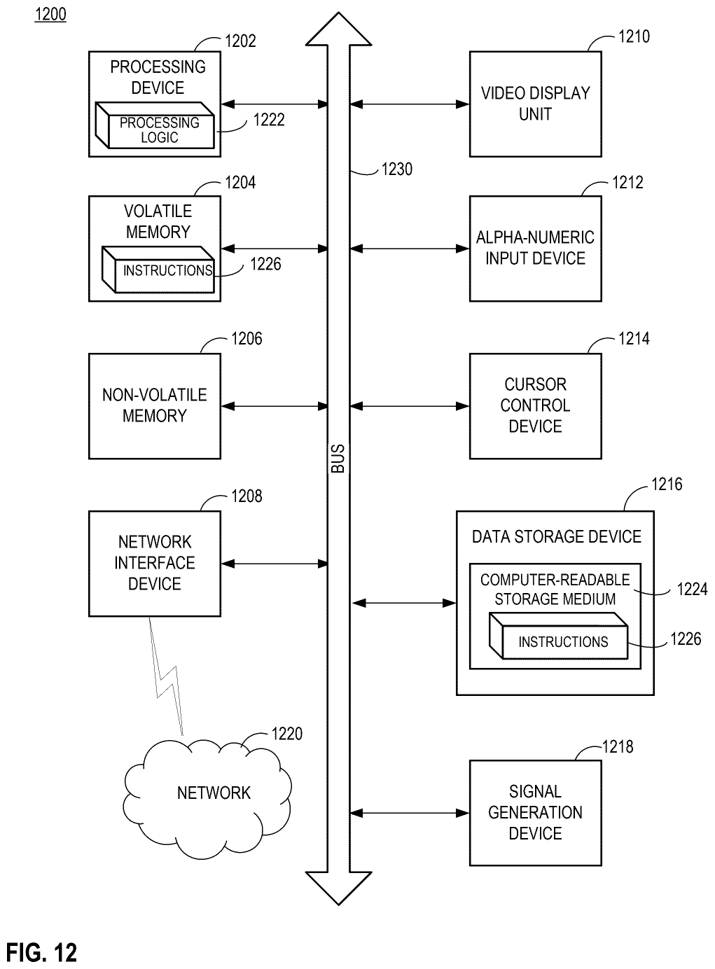

FIG. 12 illustrates an example machine of a computer system 1200 within which a set of instructions, for causing the machine to perform any one or more of the methodologies discussed herein, can be executed. In some embodiments, the computer system 1200 can be used to perform the operations of a controller (e.g., to execute an operating system to perform operations corresponding to the brace generation component 820 of FIG. 8). In alternative embodiments, the machine can be connected (e.g., networked) to other machines in a LAN, an intranet, an extranet, and/or the Internet. The machine can operate as a server or a client machine in a client-server network environment, as a peer machine in a peer-to-peer (or distributed) network environment, or as a server or a client machine in a cloud computing infrastructure or environment.

The machine can be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” should also be understood to include any collection of machines that individually or jointly execute a set (or multiple sets) of instructions to perform any one or more of the methodologies discussed herein.

The example computer system 1200 includes a processing device 1202, a main memory 1204 (e.g., read-only memory (ROM), flash memory, dynamic random access memory (DRAM) such as synchronous DRAM (SDRAM) or Rambus DRAM (RDRAM), etc.), a static memory 1206 (e.g., flash memory, static random access memory (SRAM), etc.), and a data storage system 1218, which communicate with each other via a bus 1230.

Processing device 1202 represents one or more general-purpose processing devices such as a microprocessor, central processing unit, or the like. More specifically, the processing device can be a complex instruction set computing (CISC) microprocessor, reduced instruction set computing (RISC) microprocessor, very long instruction word (VLIW) microprocessor, or a processor implementing other instruction sets, or processors implementing a combination of instruction sets. Processing device 1202 can also be one or more special-purpose processing devices such as an application-specific integrated circuit (ASIC), a field-programmable gate array (FPGA), a digital signal processor (DSP), network processor, or similar devices. The processing device 1202 is configured to execute instructions 1226 for performing the operations and steps discussed herein.

The data storage system 1218 can include a machine-readable storage medium 1224 (also known as a computer-readable medium or a non-transitory computer-readable storage medium) on which is stored one or more sets of instructions 1226 or software embodying any one or more of the methodologies or functions described herein. The instructions 1226 can also reside, completely or at least partially, within the main memory 1204 and/or within the processing device 1202 during execution by the computer system 1200, with the main memory 1204 and the processing device 1202 also constituting machine-readable storage media. In one embodiment, the processing device 1202, the network interface 1208, and the network 1220 can correspond to the computing system 800 of FIG. 8.

In one embodiment, the instructions 1226 include instructions to implement functionality corresponding to the brace generation component 820 of FIG. 8. While the machine-readable storage medium 1224 is shown in an example embodiment as a single medium, the term “machine-readable storage medium” should be understood to include a single medium or multiple media that store one or more sets of instructions. The term “machine-readable storage medium” also includes any medium capable of storing or encoding a set of instructions for execution by a machine, causing the machine to perform any one or more of the methodologies described in the present disclosure. Accordingly, the term “machine-readable storage medium” includes, but is not limited to, solid-state memories, optical media, and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm, as used herein, is generally conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. For reasons of common usage, it has proven convenient at times to refer to these signals as bits, values, elements, symbols, characters, terms, numbers, or the like.

It should be borne in mind, however, that all of these and similar terms are to be associated with the appropriate physical quantities and are merely convenient labels applied to these quantities. The present disclosure can refer to the actions and processes of a computer system, or similar electronic computing device, that manipulates and transforms data represented as physical (electronic) quantities within the computer system's registers and memories into other data similarly represented as physical quantities within the computer system's memories or registers, or other such information storage systems.

The present disclosure also relates to an apparatus for performing the operations described herein. This apparatus can be specially constructed for the intended purposes, or it can include a general-purpose computer selectively activated or reconfigured by a computer program stored in the computer. Such a computer program can be stored in a computer-readable storage medium, such as, but not limited to, any type of disk including floppy disks, optical disks, CD-ROMs, and magneto-optical disks, read-only memories (ROMs), random access memories (RAMs), EPROMs, EEPROMs, magnetic or optical cards, or any type of media suitable for storing electronic instructions, each coupled to a computer system bus.

In the above description, numerous details are set forth. However, it will be apparent to one of ordinary skill in the art, having the benefit of this disclosure, that embodiments may be practiced without these specific details. In some instances, well-known structures and devices are shown in block diagram form rather than in detail, in order to avoid obscuring the description.

Some portions of the detailed description are presented in terms of algorithms and symbolic representations of operations on data bits within a computer memory. These algorithmic descriptions and representations are used by those skilled in the data processing arts to convey the substance of their work most effectively to others skilled in the art. As used herein, an algorithm is generally conceived as a self-consistent sequence of steps leading to a desired result. The steps require physical manipulations of physical quantities. Usually, though not necessarily, these quantities take the form of electrical or magnetic signals capable of being stored, transferred, combined, compared, and otherwise manipulated. For convenience and common usage, these signals may be referred to as bits, values, elements, symbols, characters, terms, numbers, or the like.

It should be borne in mind, however, that all such terms are to be associated with the appropriate physical quantities and are merely convenient labels applied to these quantities. Unless specifically stated otherwise or as apparent from the above discussion, it is appreciated that throughout the description, discussions utilizing terms such as “determining,” “sending,” “receiving,” “scheduling,” or the like, refer to the actions and processes of a computer system or similar electronic computing device that manipulates and transforms data represented as physical (e.g., electronic) quantities within the computer system's registers and memories into other data similarly represented as physical quantities within the computer system's memories or registers, or other such information storage, transmission, or display devices.

Embodiments also relate to an apparatus for performing the operations described herein. This apparatus may be specially constructed for the required purposes, or it may comprise a general-purpose computer selectively activated or reconfigured by a computer program stored in the computer. Such a computer program may be stored in a computer-readable storage medium, including, but not limited to, any type of disk (such as floppy disks, optical disks, Read-Only Memories (ROMs), compact disc ROMs (CD-ROMs), and magneto-optical disks), Random Access Memories (RAMs), EPROMS, EEPROMs, magnetic or optical cards, or any type of media suitable for storing electronic instructions. One or more non-transitory, computer-readable storage media can have computer-readable instructions stored thereon which, when executed by one or more processing devices, cause the one or more processing devices to perform the operations described herein.

The algorithms and displays presented herein are not inherently related to any particular computer or other apparatus. Various general-purpose systems may be used with programs in accordance with the teachings herein, or it may be convenient to construct a more specialized apparatus to perform the required method steps. The required structure for a variety of these systems will appear from the description below. In addition, the present embodiments are not described with reference to any particular programming language. It will be appreciated that a variety of programming languages may be used to implement the teachings of the present embodiments as described herein. It should also be noted that the terms “when” or the phrase “in response to,” as used herein, should be understood to indicate that there may be intervening time, intervening events, or both before the identified operation is performed.

It is to be understood that the above description is intended to be illustrative, and not restrictive. Many other embodiments will be apparent to those of skill in the art upon reading and understanding the above description. The scope of the present embodiments should, therefore, be determined with reference to the appended claims, along with the full scope of equivalents to which such claims are entitled.

What is claimed is:

1. A brace comprising:
a plurality of first helix structures that wind in a first direction from a proximal end of the brace to a distal end of the brace, configured to conform to a contour of an object; and
a plurality of second helix structures that wind in a second direction from the proximal end to the distal end, conforming to the contour of the object, wherein each corresponding first helix structure of the plurality of first helix structures is rotatably coupled to a corresponding second helix structure of the plurality of second helix structures to form a corresponding helix structure pair of a plurality of helix structure pairs, and wherein the first direction opposes the second direction.

2. The brace of claim 1, wherein the corresponding helix structure pair is rotatably coupled using a corresponding pin-and-hole connection that provides rotation of a first helix structure of the plurality of first helix structures and a second helix structure of the plurality of second helix structures about the corresponding pin-and-hole connection.

3. The brace of claim 1, wherein overlapping of the plurality of first helix structures and the plurality of second helix structures forms a crosshatched configuration between the proximal end and the distal end, forming a plurality of openings.

4. The brace of claim 3, wherein a distance between a proximal opening end and a distal opening end of each opening of the plurality of openings decreases in response to axial compression of the brace.

5. The brace of claim 3, wherein a distance between a proximal opening end and a distal opening end of each opening of the plurality of openings increases in response to axial extension of the brace.

6. The brace of claim 1, wherein the object is at least one of: a body part, a prosthetic limb, a medical device, sports equipment, a tool, a package, or an electronic device.

7. The brace of claim 1, wherein the plurality of first helix structures and the plurality of second helix structures each conform to the contour of the object by:
determining the contour of the object to be braced;
selecting dimensions for the plurality of first helix structures and the plurality of second helix structures based on the object;
configuring the plurality of first helix structures and the plurality of second helix structures to follow the contour by overlapping the plurality of first helix structures and the plurality of second helix structures at regions along the plurality of first helix structures and the plurality of second helix structures;
adjusting an orientation of the plurality of first helix structures and the plurality of second helix structures along the contour of the object; and
operatively coupling the plurality of first helix structures and the plurality of second helix structures to maintain their arrangement relative to at least a portion of the contour of the object.

8. The brace of claim 1, wherein the plurality of first helix structures and the plurality of second helix structures comprise one or more of: a polymer, a metal, a ceramic, a fabric, a natural fiber, a synthetic fiber, or an elastomer.

9. The brace of claim 1, wherein the plurality of first helix structures and the plurality of second helix structures comprise one or more of: a thermopolymer additive, a waterproof additive, a temperature resistance additive, an antibacterial additive, an antimicrobial additive, an ultraviolet (UV) additive, or a colorant.

10. The brace of claim 1, wherein the plurality of first helix structures and the plurality of second helix structures comprise a plurality of units that comprise one or more of particles, granules, extrudates, fibers, or powders.

11. A method comprising:
determining a contour of an object to be braced;
selecting dimensions for a plurality of first helix structures and a plurality of second helix structures of a brace based on the object;
configuring the plurality of first helix structures and the plurality of second helix structures to follow the contours by overlapping the plurality of first helix structures and the plurality of second helix structures at regions along the plurality of first helix structures and the plurality of second helix structures;
adjusting an orientation of the plurality of first helix structures and the plurality of second helix structures along the contour of the object; and
operatively coupling the plurality of first helix structures and the plurality of second helix structures to maintain their arrangement relative to at least a portion of the contour of the object.

12. The method of claim 11, wherein each first helix winds in a first direction from a proximal end of the brace to a distal end of the brace configured to conform to the contour of the object.

13. The method of claim 11, wherein each second helix winds in a second direction from a proximal end of the brace to a distal end of the brace, conforming to the contour of the object.

14. The method of claim 11, wherein the corresponding helix structure pair is rotatably coupled using a corresponding pin-and-hole connection that provides rotation of a first helix structure of the plurality of first helix structures and a second helix structure of the plurality of second helix structures about the corresponding pin-and-hole connection.

15. The method of claim 11, wherein the object is at least one of: a body part, a prosthetic limb, a medical device, sports equipment, a tool, a package, or an electronic device.

16. The method of claim 11, wherein overlapping of the plurality of first helix structures and the plurality of second helix structures forms a crosshatched configuration between the proximal end and the distal end, forming a plurality of openings.

17. The method of claim 16, wherein a distance between a proximal opening end and a distal opening end of each opening of the plurality of openings decreases in response to axial compression of the brace.

18. The method of claim 16, wherein a distance between a proximal opening end and a distal opening end of each opening of the plurality of openings increases in response to axial extension of the brace.

19. The method of claim 11, wherein the plurality of first helix structures and the plurality of second helix structures comprise one or more of: a polymer, a metal, a ceramic, a fabric, a natural fiber, a synthetic fiber, or an elastomer.

20. The method of claim 11, wherein the plurality of first helix structures and the plurality of second helix structures comprise one or more of: a thermopolymer additive, a waterproof additive, a temperature resistance additive, an antibacterial additive, an antimicrobial additive, an ultraviolet (UV) additive, or a colorant.
